(19) United States
(12) Patent Application Publication
(10) Pub. No.: US 2026/0151155 A1
(43) Pub. Date: Jun. 4, 2026

(54) APPARATUS AND METHODS FOR TRIGGER DIGIT RELEASE

(71) Applicant: Alton Lee Hunter, JR., Columbia, TN (US)

(72) Inventor: Alton Lee Hunter, JR., Columbia, TN (US)

(21) Appl. No.: 19/280,470

(22) Filed: Jul. 25, 2025

Related U.S. Application Data
(60) Provisional application No. 63/675,340, filed on Jul. 25, 2024.

(51) Int. Cl. A61B 17/32; A61B 17/00

(52) U.S. Cl. CPC A61B 17/320016; A61B 2017/0023; A61B 2017/00424; A61B 2017/320052

(57) ABSTRACT

A surgical tool apparatus is provided. The apparatus may a handle, a shaft extending distally from the handle, a receiver defined in a proximal end of the handle opposite the shaft, a hub disposed in the receiver, and a foot disposed on a distal end of the shaft opposite the handle. The foot may include an opening. The apparatus may further include a channel defined from the receiver to the opening, and a cutting instrument disposed in the channel. The cutting instrument may be fixed to the hub, and include a tip that is configured to dissect tissue during a surgical procedure. Some or all of the apparatus may be made of a disposable material, such as plastic or a polymer material.

### CROSS-REFERENCE TO RELATED APPLICATION

This application is a non-provisional of and claims priority to U.S. Provisional Application No. 63/675,340, filed Jul. 25, 2024, entitled “APPARATUS AND METHODS FOR TRIGGER DIGIT RELEASE,” which is hereby incorporated by reference in its entirety.

### BACKGROUND

The present disclosure relates generally to devices and methods for performing surgery and more particularly to devices and methods for dissecting tissue in a human hand.

Finger and thumb tendonitis, also known as trigger finger or trigger digit, is one of the most common orthopedic conditions. Trigger digit occurs when a finger or thumb is unable to fully flex and extend relative to the hand. In many cases, the finger or thumb becomes partially bent toward the palm of the hand, and a patient is unable to straighten the digit. Trigger digit causes pain and stiffness in the hand and fully disables a patient's ability to use the digit in some cases.

In cases of trigger digit, the flexor tendon that causes a finger or thumb to flex is unable to freely travel through a corresponding tendon sheath or a tendon pulley. In some instances, the A1 pulley in a patient's hand becomes inflamed or thickened, or the flexor tendon itself becomes inflamed or thickened, thereby restricting travel of the flexor tendon and preventing proper flexion or extension of a digit.

Trigger digit release is one of the most common treatments provided by orthopedic surgeons. Treatment options for trigger finger or trigger thumb include splinting (rarely successful), physical therapy (almost never successful), cortisone injections (less than 50% success rate) and surgery. Percutaneous trigger digit releases are performed in clinical offices by surgeons in an office setting, saving the patient the inconvenience and expense of a formal “open” surgical procedure. Historically, percutaneous trigger digit releases have been performed in clinical offices by a select few orthopedic surgeons, using a variety of techniques with a highly variable success rate (at best) of 90% with regards to eliminating the mechanical symptoms of “catching” in the digit and pain. Traditional “open” surgery has a success rate of better than 99%, thus the vast majority of trigger digit release operations are performed in this manner, requiring a surgical facility along with the added expense and inconvenience associated with it.

Trigger digit can be treated in many cases by severing, or dissecting, the A1 pulley to free the affected flexor tendon, known as a trigger digit release operation. Trigger digit release surgery can be performed in some instances by percutaneously inserting a needle (e.g., a spinal needle, or other suitable needle) into the palm of the hand and using the sharpened tip of the needle as a cutting tool to sever the A1 pulley of the affected digit. Such operations may be performed in a doctor's office without general anesthesia. However, conventional methods of percutaneous trigger digit release using a needle may be difficult for some surgeons to perform because the needle tip cannot be directly observed in real time during the operation, and many surgeons are not comfortable guiding the needle tip to the A1 pulley percutaneously based on feel. For this reason, most surgeons choose to perform trigger release operations in open surgery, forming an incision in the palm to gain direct access to and visual observation of the A1 pulley. However, such operations must be performed in a surgical suite and may require additional anesthesia. This leads to much greater expense and a longer recovery time for the patient.

What is needed are improvements in devices and methods for percutaneous trigger digit release such that operations may be performed safely, reliably, and repeatably in a surgeon's office as opposed to a surgical suite.

### BRIEF SUMMARY

The present disclosure provides a surgical tool for dissecting, or cutting, tissue. In some embodiments, the surgical tool is configured for performing an operation to treat trigger digit in a finger or thumb by dissecting the A1 pulley to free a constrained flexor tendon. The surgical tool operates as a guide for steering a needle to safely, precisely, and reliably dissect tissue by a surgeon in either a clinical office or in a surgical suite.

In some embodiments, the surgical tool provides an apparatus including a handle, a shaft extending distally from the handle, a receiver defined in a proximal end of the handle opposite the shaft, a hub disposed in the receiver, and a foot disposed on a distal end of the shaft opposite the handle. The foot may include an opening. The apparatus may further include a channel defined from the receiver to the opening, and a cutting instrument disposed in the channel. The cutting instrument may be fixed to the hub, and include a tip that is configured to dissect tissue during a surgical procedure. Thus, a tip of the needle may protrude from the shaft distal to the foot such that the foot may be inserted in a small incision and the tip of the needle may be precisely advanced to dissect tissue by manually controlling the handle in a back and forth or side to side motion.

In some embodiments, the foot includes a concave contact surface shaped to accommodate (e.g., align with) a tendon sheath. The opening may be disposed on the concave contact surface. The concave contact surface and a rear surface of the handle on the proximal end of the handle may be parallel. In some cases, each of the concave contact surface and the rear surface form a seventy-degree angle relative to a longitudinal axis of the shaft. In further embodiments, the concave contact surface is curved, such that the concave contact surface forms a first radius of curvature, and the rear surface of the handle forms is curved, such that the rear surface forms a second radius of curvature. The first radius of curvature and the second radius of curvature may be equivalent.

In some embodiments, the apparatus further includes a biasing member disposed in the receiver between the hub and the handle. The biasing member may resist advancement of the hub. Additionally, lateral ends of the foot may extend distally relative to the opening. In this sense, when the hub is advanced toward the foot, the tip of the cutting instrument may project distally out of the opening of the foot but be prevented from extending further from the opening than the opposing lateral ends of the foot. Accordingly, the foot may serve as a soft tissue dissector tool, retractor tool, and a safety mechanism by limiting the depth of the deployed cutting instrument and thus preventing damage to a flexor tendon.

Further embodiments of the present disclosure include a method of performing surgery. The method may include providing the aforementioned handle, shaft, foot, cutting instrument, and hub. The method may further include positioning the foot on the tendon sheath and advancing the hub, such that the tip of the cutting instrument projects distally out of the opening and is operable to dissect tissue during the surgical procedure. Thus, once aligned with an A1 pulley, the tip of the cutting instrument may be used to cut the tissue at a desired location. The entire procedure may be performed with or without ultrasound imaging in a doctor's office.

Accordingly, objects of the present disclosure may include: (a) providing an effective, predictable, safe and technically easy percutaneous trigger digit release tool; (b) providing a trigger digit release tool compatible for use with ultrasound imaging in a doctor's office as opposed to open surgery; (c) providing a trigger digit release tool deployable with a small incision that does not require suturing; and (d) providing a trigger digit release tool with a foot that limits depth of cut to limit potential damage to the flexor tendon.

Further objects of the present disclosure may include: (a) providing a trigger digit release tool with a bidirectional working end that may be utilized using a push or a pull cutting technique; (b) providing a trigger digit release tool with a foot having one or more inclined edges to dissect tissue while advancing; (c) providing a trigger digit release tool to utilize a spinal needle deployed as a cutting instrument having precise depth control by manipulating the position of the needle relative to the tool; (d) providing a trigger digit release tool having a handle with curved upper surface and bottom surface to approximate the curvature of a flexor sheath to facilitate precise orientation and visualization of the cutting instrument relative to the anatomy of the hand; and (e) providing a trigger digit release tool that is sterile and disposable.

Numerous other objects, advantages and features of the present disclosure will be readily apparent to those of skill in the art upon a review of the following drawings and description.

### BRIEF DESCRIPTION OF THE FIGURES

FIG. 1 illustrates a rear perspective view of an embodiment of a surgical tool.

FIG. 2 illustrates a front perspective view of an embodiment of a surgical tool.

FIG. 3 illustrates a side cross-sectional view of an embodiment of a surgical tool.

FIG. 4 illustrates a side cross-sectional view of an embodiment of a body of a surgical tool.

FIG. 5 is a front view of an embodiment of a body of a surgical tool.

FIG. 6 is a rear view of an embodiment of a surgical tool.

FIG. 7 is a partial top view of an embodiment of a body of a surgical tool.

FIG. 8 is a side view of an embodiment of a body of a surgical tool.

FIG. 9 is a top view of an embodiment of a body of a surgical tool.

FIG. 10 is a bottom view of an embodiment of a body of a surgical tool.

FIG. 11 is a partial side cross-sectional view of an embodiment of a body of a surgical tool.

FIG. 12 a top perspective view of an embodiment of a shaft and a foot of a surgical tool.

FIG. 13 is a top view of an embodiment of a shaft and a foot of a surgical tool.

FIG. 14 is a side cross-sectional view of an embodiment of a shaft and a foot of a surgical tool.

FIG. 15 is a rear perspective view of an embodiment of a body of a surgical tool.

FIG. 16 is a front perspective view of an embodiment of a hub of a surgical tool.

FIG. 17 is a front perspective view of an embodiment of a hub and a cutting instrument of a surgical tool.

FIG. 18 is a rear perspective view of an embodiment of a handle of a surgical tool.

FIG. 19 is a rear perspective view of an embodiment of a handle of a surgical tool with a spring.

FIG. 20 is a side cross-sectional view of a an embodiment of a surgical tool prior to advancing a cutting instrument of the surgical tool.

FIG. 21 is a first partial side cross-sectional view of an embodiment of a surgical tool prior to advancing a cutting instrument of the surgical tool.

FIG. 22 is a second partial side cross-sectional view of an embodiment of a surgical tool prior to advancing a cutting instrument of the surgical tool.

FIG. 23 is a side cross-sectional view of an embodiment of a surgical tool after advancing a cutting instrument of the surgical tool.

FIG. 24 is a first partial side cross-sectional view of an embodiment of a surgical tool after advancing a cutting instrument of the surgical tool.

FIG. 25 is a second partial side cross-sectional view of an embodiment of a surgical tool after advancing a cutting instrument of the surgical tool.

FIG. 26 is a top perspective view of an embodiment of a shaft and a foot of a surgical tool after advancing a cutting instrument of the surgical tool.

FIG. 27 is a top view of an embodiment of a shaft and a foot of a surgical tool after advancing a cutting instrument of the surgical tool.

FIG. 28 is a side perspective view of an embodiment of a cutting instrument of a surgical tool.

FIG. 29 is a partial side view of an embodiment of a cutting instrument of a surgical tool.

FIG. 30 is a front view of an embodiment of a cutting instrument for a surgical tool.

FIG. 31 illustrates an exemplary embodiment of a method of treating a trigger digit, including the step of making a small incision in a hand.

FIG. 32 illustrates an exemplary embodiment of a method of treating a trigger digit, including the step of positioning a foot of a surgical tool over a tendon sheath.

FIG. 33 illustrates an exemplary embodiment of a method of treating a trigger digit, including the step of advancing an instrument for cutting tissue through the cutting tool.

FIG. 34 illustrates an exemplary embodiment of a method of treating a trigger digit, including the step of using a cutting instrument installed in a surgical tool to cut diseased tissue, in this case a soft tissue pulley.

FIG. 35 illustrates a detailed exemplary embodiment of a method of treating a trigger digit, including the step of using a cutting instrument installed in a surgical tool to cut diseased tissue, in this case a soft tissue pulley.

FIG. 36 is a side perspective view of an embodiment of a shaft and a foot of a surgical tool.

FIG. 37 is a bottom perspective view of an embodiment of a shaft and a foot of a surgical tool.

### DETAILED DESCRIPTION

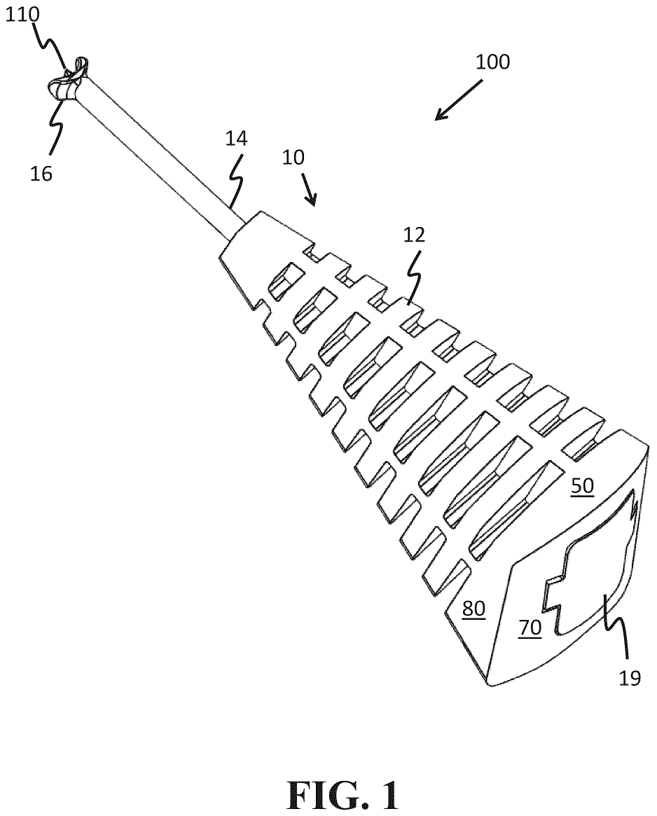

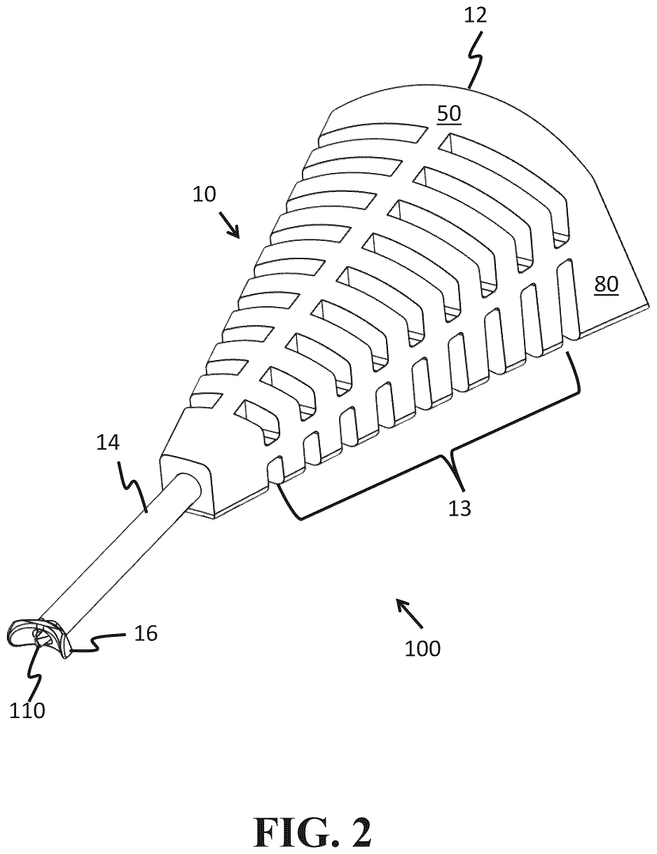

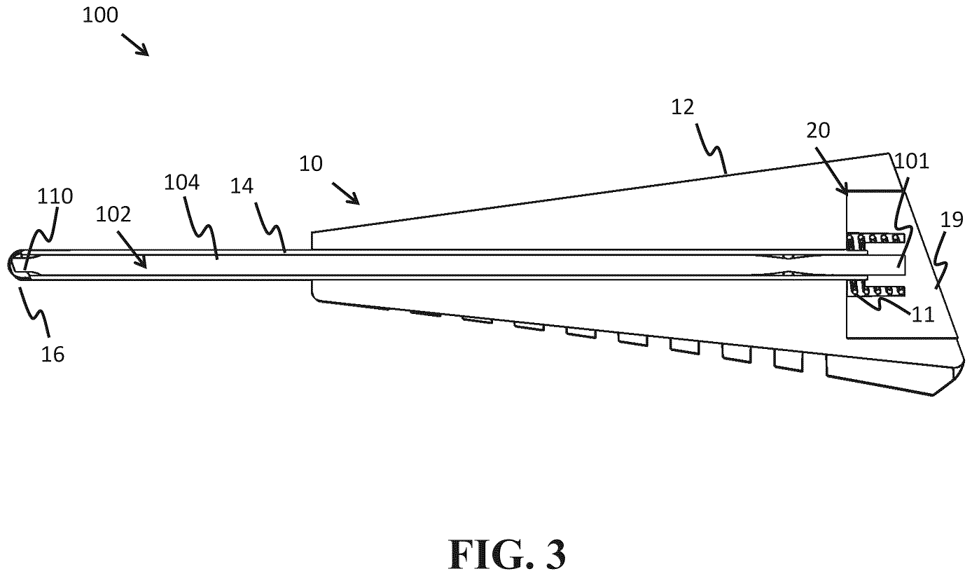

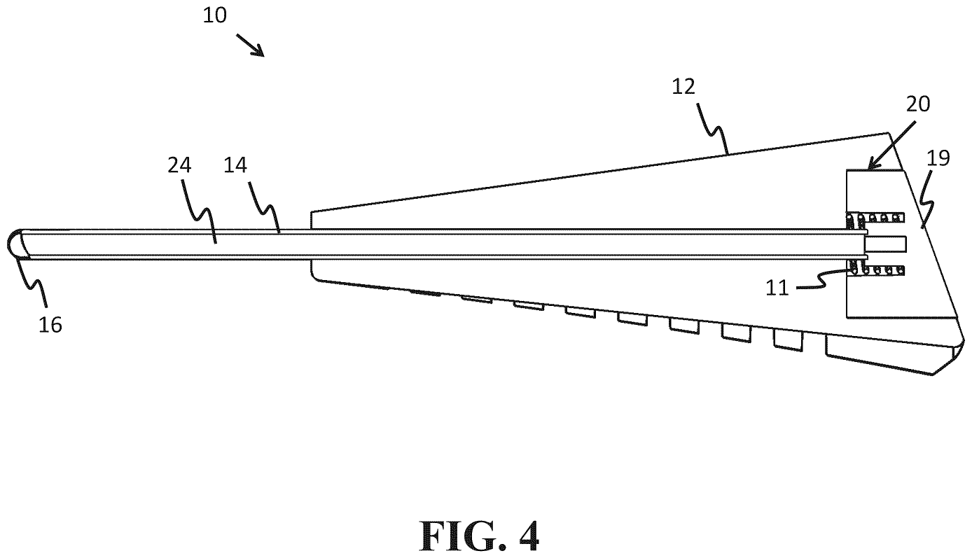

The present disclosure provides a tissue dissection apparatus and methods for treatment of trigger digit. Referring to FIGS. 1-4, in some embodiments, a surgical tool apparatus (apparatus) 100 includes a body 10 and, as shown with particular reference to FIG. 3, a cutting instrument 102. As shown with particular reference to FIG. 4, the body 10 may include a handle 12, a shaft 14, and a foot 16. The body 10 may be configured such that the handle 12 can be held in a user's hand with the shaft 14 extending toward an incision site for performing a trigger digit release surgical procedure in a surgeon's office. In some embodiments, the apparatus 100 includes any suitable rigid material for guiding the cutting instrument 102 during surgery. In some embodiments, the apparatus 100 includes a disposable material, such as plastic. In other embodiments, the apparatus 100 includes a polymer material. In some embodiments, the cutting instrument 102 is made of metal or some other material suitably rigid for cutting tissue. In some embodiments, the cutting instrument 102 is made of metal or some other tissue-cutting material, while some or all of the body 10 (e.g., the handle 12, the shaft 14, and/or the foot 16, or any suitable components of the apparatus 100) is made of the aforementioned disposable material (e.g., plastic, a polymer material, etc.) Of course, it should be appreciated that while the cutting instrument 102 may be made of a material suitable for cutting tissue, the apparatus 100 in its entirety may nonetheless be suitable for disposable use. Depending on the implementation, the apparatus 100 may be made of any material such that, during a surgical procedure utilizing real-time ultrasound imaging, the procedure may readily identify the precise location of the cutting instrument 102 due to the imaging contrast between the material of the cutting instrument 102 and the other material of the apparatus 100 (e.g., the body 10).

The shaft 14 may extend or project distally from the handle 12 and, as shown with particular reference to FIG. 4, form a channel 24. The shaft 14 may be a straight tube-shaped member. In this sense, the channel 24 may be defined through the handle 12 and at least partially through the shaft 14. In other words, the shaft 14 may include a member extending from handle 12 to accommodate passage of the cutting instrument 102 through the channel 24 of the shaft 14. In other embodiments, the shaft 14 is a tube-shaped member having a curvilinear profile. In any case, the cutting instrument 102 may be disposed in the channel 24. In this sense, the cutting instrument 102 may be axially received by the body 10 of the apparatus 100, such that the cutting instrument 102 is configured to be passed through the channel 24 and project from the shaft 14 to make an incision in tissue. Thus, the channel 24 may be configured to receive the cutting instrument 102.

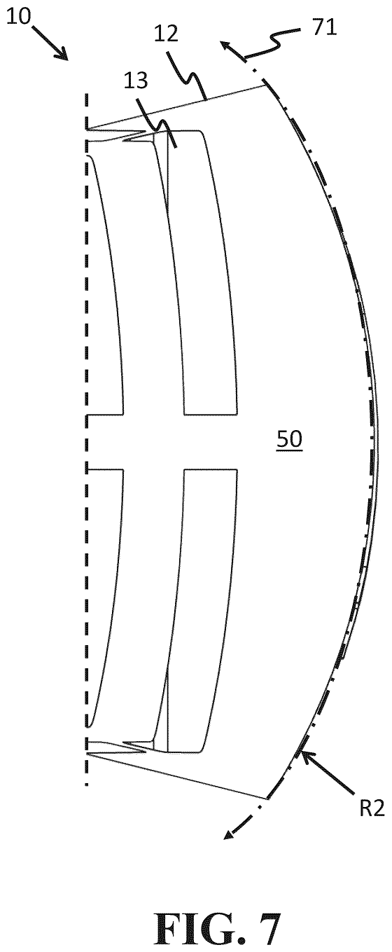

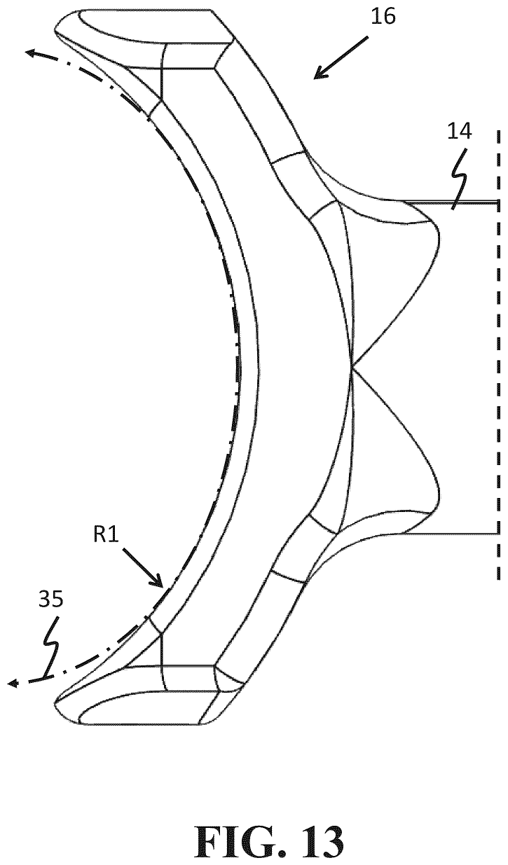

The foot 16 may be disposed on a distal end of the shaft 14 opposite the handle 12. As discussed in greater detail below with reference to FIGS. 7 and 10-13, the foot 16 may include a concave contact surface 36 shaped to accommodate (e.g., align with) a tendon sheath. The concave contact surface 36 may aid in centering the foot 16 on the tendon sheath and sweeping soft tissue off the tendon sheath. In some embodiments, the concave contact surface 36 forms a first radius of curvature R1. A rear surface 70 of the handle 12 (shown with reference to FIG. 1) may also be curved, such that the rear surface 70 forms a second radius of curvature R2. The first radius of curvature R1 and the second radius of curvature R2 may be equivalent. In other words, the concave contact surface 36 and the rear surface 70 may have similar curvature.

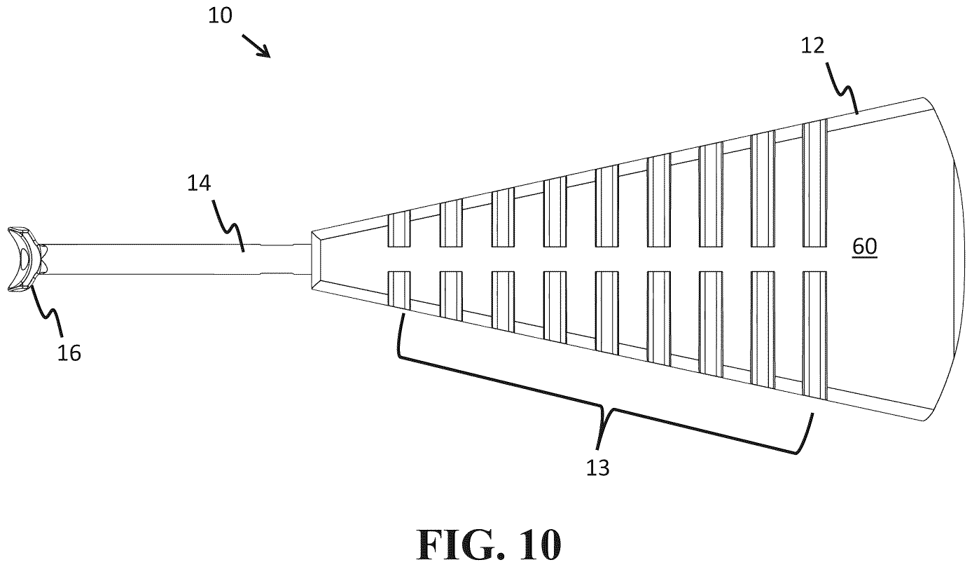

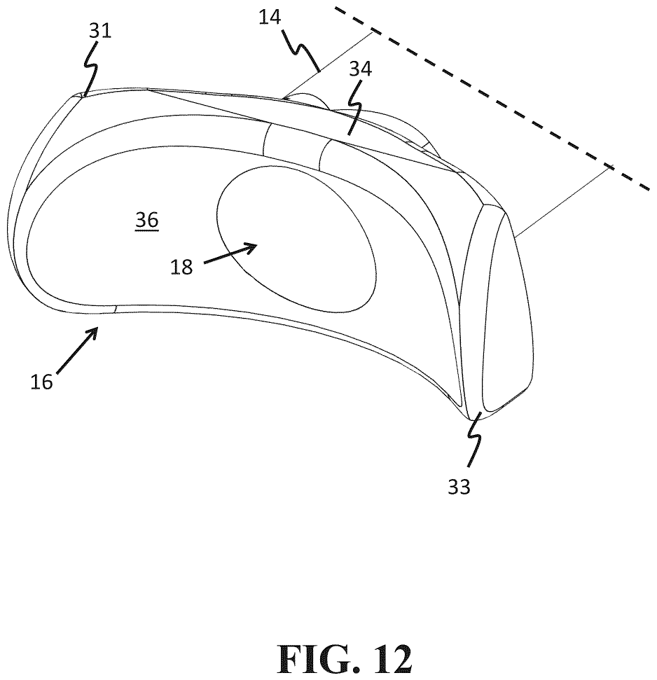

In some embodiments, and as shown with particular reference to FIG. 3, the handle 12 includes a receiver 20. The receiver 20 may be defined in a proximal end of the handle 12 opposite the shaft 14. As discussed in greater detail below with reference to FIGS. 10 and 12, the foot 16 may include an opening 18. In this sense, the channel 24 may be defined from the receiver 20 to the opening 18 of the foot 16. In some embodiments, the apparatus 100 or, more particularly, the body 10, further includes a hub 19. The hub 19 may be disposed in the receiver 20. In turn, the cutting instrument 102 maybe fixed (e.g., glued or welded) to the hub 19. In some embodiments, the hub 19 is made of a disposable material, such as plastic or a polymer material.

In some embodiments, and as shown with particular reference to FIG. 3, the cutting instrument 102 includes a needle having a tube 104 (e.g., needle tube, a rod, a needle rod, a k-wire, etc.) extending between a proximal end 101 and a tip 110 on a distal end of the tube 104. For example, the tip 110 may be configured to dissect tissue during a surgical procedure. In this sense, the tip 110 may protrude from the shaft 14 distal to the foot 16 such that the foot 16 may be inserted in a small incision and the tip 110 may be precisely advanced to dissect tissue by manually controlling the handle 12 in a back and forth or side to side motion. In various other embodiments, the cutting instrument 102 may be any suitable instrument to be passed through the channel 24 and project from the shaft 14 to make an incision in tissue. In some embodiments, the cutting instrument 102 includes a spinal needle. In embodiments where the shaft 14 includes a straight tube-shaped member, the tube 104 of the cutting instrument 102 may be straight.

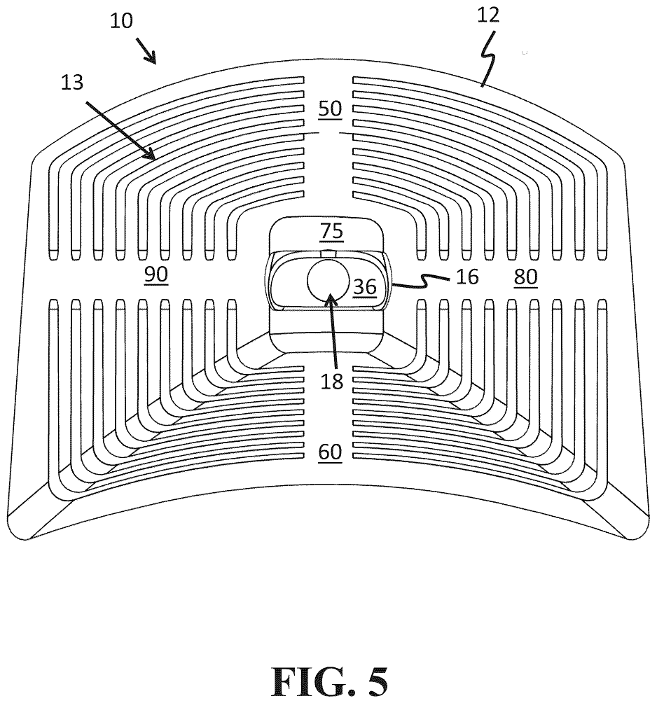

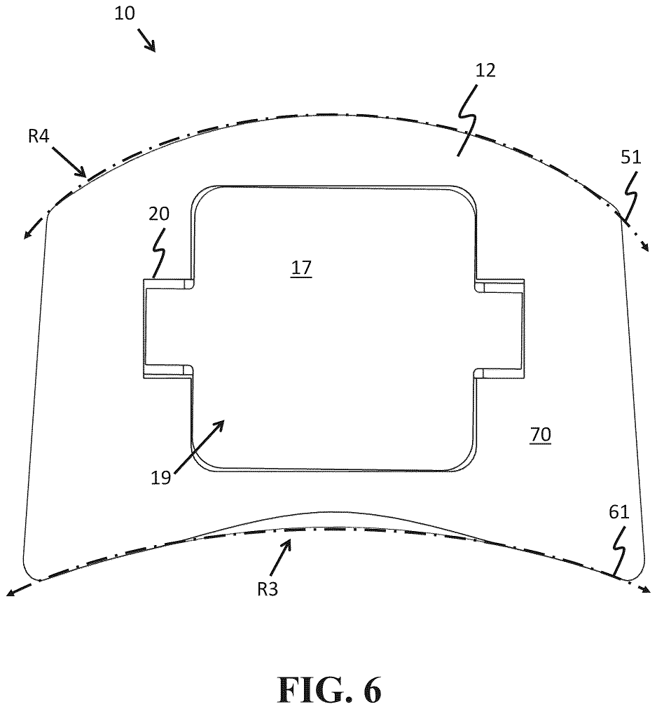

Referring now to FIG. 5, a front side of the body 10 is shown, according to some embodiments of present disclosure. The handle 12 of the body 10 may include a top surface 50, a bottom surface 60, a left surface 80, and a right surface 90. The top, bottom, left, and right surfaces 50, 60, 80, and 90 may narrow in width from the rear surface 70 (on the proximal end of the handle 12, and as shown with particular reference to FIG. 6) to a front surface 75, from which the shaft 14 may project. For example, the handle 12 may include a member having a profile dimensioned such that handle 12 may be gripped in a user's hand. As mentioned above, the handle 12 may be made of a disposable material, such as plastic or a polymer material.

Referring now to FIG. 6, a back side of the body 10 is shown, according to some embodiments of the present disclosure. The top surface 50 of the handle 12 may include a convex curved surface, and the bottom surface 60 of the handle 12 may include a concave curved surface. In some embodiments, the bottom surface 60 includes a third radius of curvature R3, and the top surface 50 includes a fourth radius of curvature R4. For example, the top surface 50 may form a top surface plane 51 that defines the third radius of curvature R3, and the bottom surface 60 may form a bottom surface plane 61 that defines the fourth radius of curvature R4. In some embodiments, the third radius of curvature R3 and/or the fourth radius of curvature R4 form a thirty-degree arc.

Referring now to FIG. 7, a rear top side of the body 10 is shown, according to some embodiments of the present disclosure. As mentioned above, the handle 12 of the body 10 may include the rear surface 70. The rear surface 70 may define a rear surface plane 71. The rear surface plane 71 formed by the rear surface 70 may define the second radius of curvature R2. In some embodiments, the second radius of curvature R2 forms a thirty-degree arc.

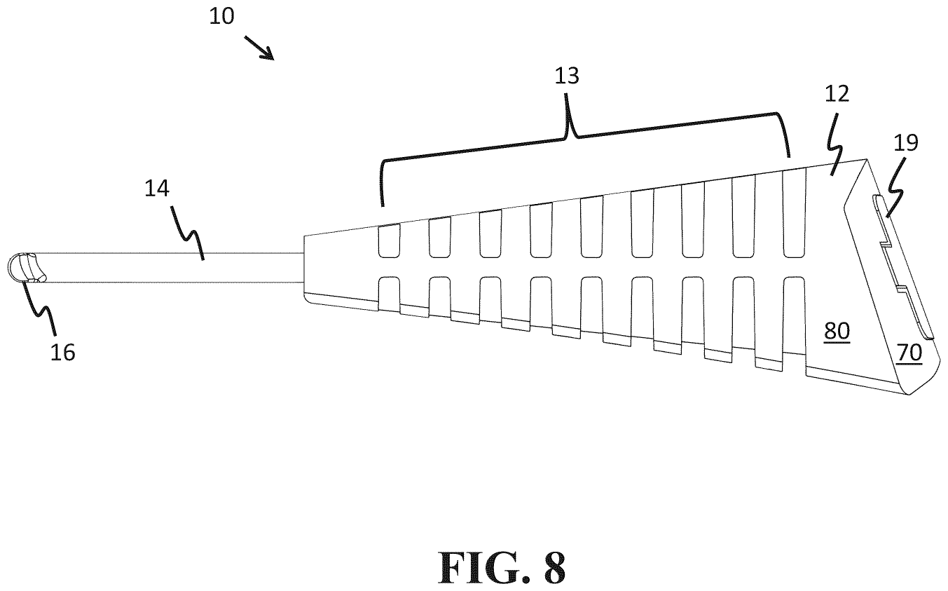

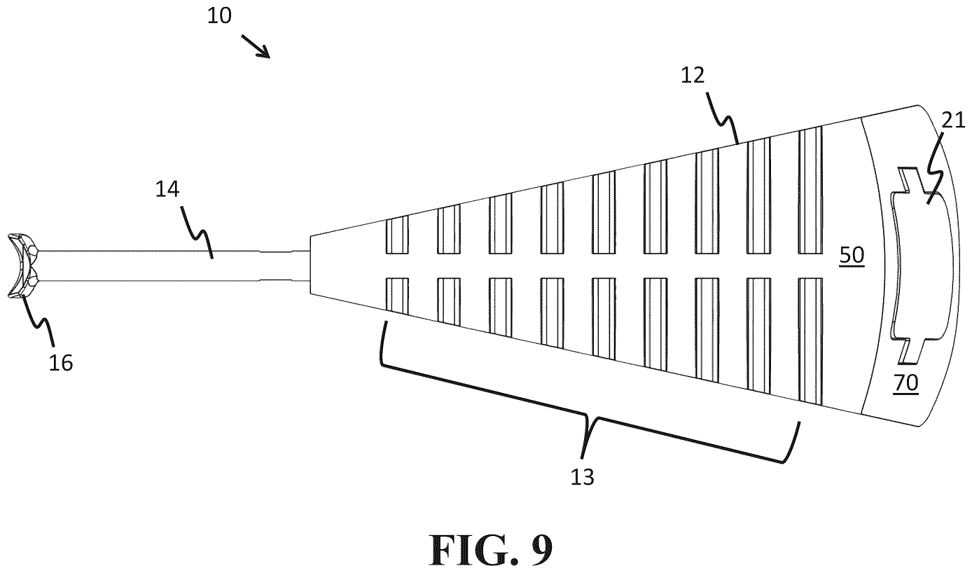

Referring now to FIGS. 8-10, side, top, and bottom views of the body 10 are shown, according to some embodiments of the present disclosure. The handle 12 of the body 10 may include a series of ribs 13. The ribs 13 may be spaced longitudinally along the length of the handle 12. Advantageously, particularly in the context of providing the body 10 as a disposable component, the ribs 13 may decrease the amount of material required to provide the handle 12, and may otherwise facilitate easier manufacturing (e.g., molding) of the handle 12, and therefore the apparatus 100 in its entirety. Depending on the implementation, the handle 12 may include a handle length 40 of about forty millimeters, a handle width 43 of about thirty millimeters, and a handle height 46 of about thirty millimeters. The handle 12 may include other shapes and dimensions in further embodiments.

As mentioned above, the handle 12 may include the receiver 20 on the handle 12. The receiver 20 may include a recess formed in handle 12, which may be shaped and dimensioned to accommodate the hub 19. In this sense, receiver 20 may be integrally formed as a recess in handle 12. As discussed in greater detail below, the hub 19 may be advanced by a user (e.g., towards the foot 16) in order to advance the instrument 102 through the channel 24, such that the tip 110 of the instrument 102 projects distally out of the opening 18 of the foot 16 to make an incision in the A1 pulley.

As mentioned above, the shaft 14 extends distally from the handle 12, according to some embodiments of the present disclosure. Of course, depending on the implementation, the shaft 14 may take any other suitable shape providing an elongated protrusion distal to the handle 12 in order to provide the advantages discussed herein. In some embodiments, the shaft 14 includes a length of about forty-five millimeters. In some embodiments, the shaft 14 is made of a disposable material, such as plastic. In other embodiments, the shaft 14 may be made of a rigid material sufficient to maintain its shape when a resilient instrument, such as a metal needle, is passed through the channel 24.

In some embodiments, the shaft 14 is integrally formed with the handle 12 in a one-piece construction. In other embodiments, the shaft 14 includes a separate component mechanically attached to the handle 12 at a joint between the shaft 14 and the handle 12. Thus, the shaft 14 may be a separate component that extends through the handle 12 (e.g., from the receiver 20 to the front surface 75 of the handle 12 (shown with reference to FIG. 5)), and projects from the handle 12 (e.g., from the front surface 75 of the handle 12). In other embodiments, the portion of the shaft 14 projecting free of the front surface 75 of the handle 12 is a separate components mechanically attached to the handle 12 at a joint located near the front surface 75, and the portion of the shaft 14 housed within the handle 12 is integrally formed with the handle 12 in one-piece construction. In further embodiments, the body 10 does not include the shaft 14. For instance, the foot 16 may simply be disposed on or otherwise connected to a distal end of the handle 12. In any case, the channel 24 may be defined from the port 22 to the opening 18 of the foot 16. Thus, the channel 24 may be defined through the handle 12 and at least partially through the shaft 14 in order to accommodate passage of the cutting instrument 102 therethrough. The channel 24 may extend through the shaft 24 toward the distal end of the shaft 14.

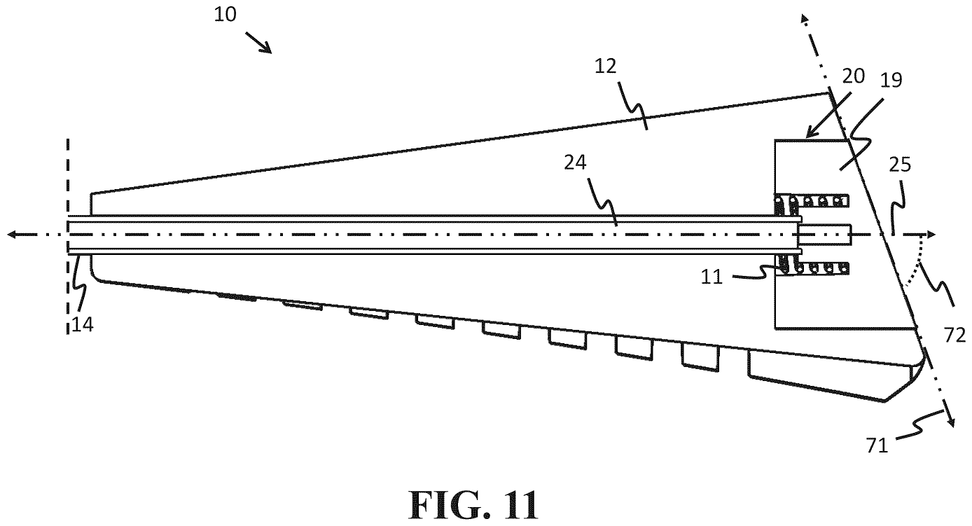

Referring to FIG. 11, a partial side cross-sectional view of the body 10 is shown, according to some embodiments of the present disclosure. As shown, the channel 24 may define a longitudinal axis 25 that is straight (or substantially straight) and extends from the receiver 20 to the opening 18 of the foot 16. As mentioned above, the rear surface 70 of the handle 12 may form the rear surface plane 71. In some embodiments, the aforementioned rear surface plane 71 forms a rear surface angle 72 relative to the longitudinal axis 25 of the channel 24 (or, more generally, relative to the shaft 14). Depending on the implementation, the rear surface angle 72 may be between about forty-five and about ninety degrees. In some embodiments, the rear surface angle 72 is between about sixty and about eighty degrees. In further embodiments, the rear surface angle 72 is about seventy degrees.

Referring now to FIGS. 12-14, the foot 16 is shown in greater detail, according to some embodiments of the present disclosure. As mentioned above, the foot 16 may include the concave contact surface 36. The opening 18 of the foot 16 may be disposed on the concave contact surface 36. The contact surface 36 may be dimensioned to approximate the surface contour of a flexor tendon sheath, thereby allowing foot 16 to slide along the surface of the flexor tendon sheath during a procedure. The concave contact surface 36 may form a contact surface plane 35 that defines the first radius of curvature R1. As mentioned above, the rear surface plane 71 formed by the rear surface 70 may define the second radius of curvature R2.

In some embodiments, each of the first radius of curvature R1 and the second radius of curvature R2 form about a thirty-degree arc. In this sense, when the foot 16 is positioned on the tendon sheath as discussed above, the equivalent arcs formed by the concave contact surface plane 35 and the rear surface plane 71 (forming equivalent radii of curvature R1, R2, respectively), may allow a user to readily perceive the orientation of the channel 24, and thus the tip 110. This configuration may aid a surgeon in ensuring concise control and proper alignment of the apparatus 100 during a procedure.

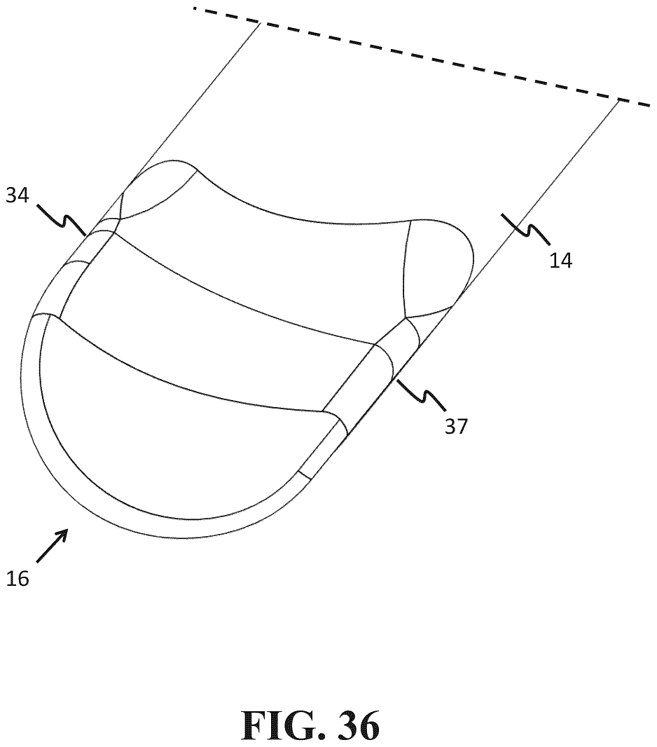

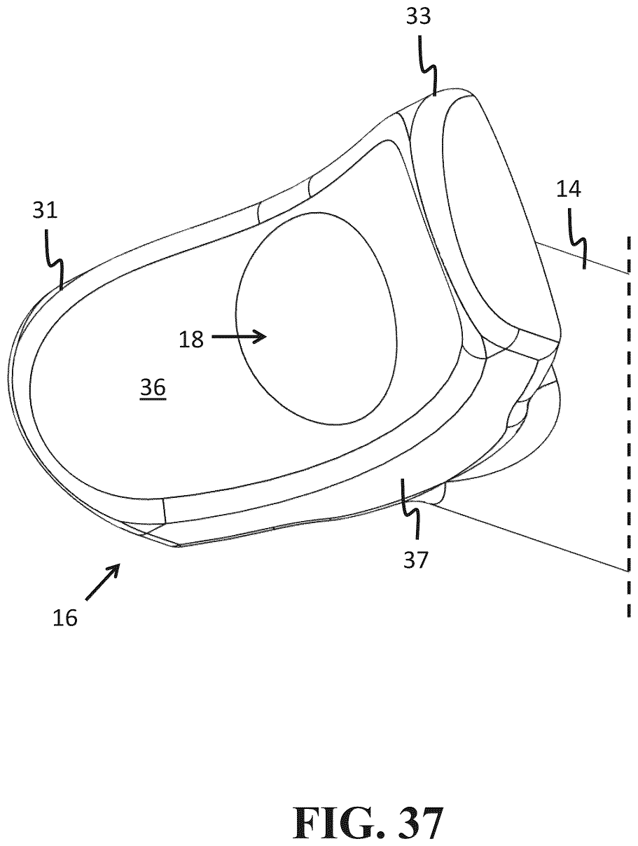

Referring particularly to FIG. 12, and with additional reference to FIGS. 36 and 37, in some embodiments the foot 16 includes a leading edge ramp 34 and a trailing edge ramp 37. Each of the leading and trailing edge ramps 34, 37 may provide an inclined surface that operates to lift tissue off the tendon sheath as the foot 16 travels relative to the tendon sheath, thereby providing enhanced access to the A1 pulley with the tip 110 to perform a dissection of the pulley. Each of the leading and trailing edge ramps 34, 37 may include a flat inclined profile, or a curved inclined profile, in some embodiments.

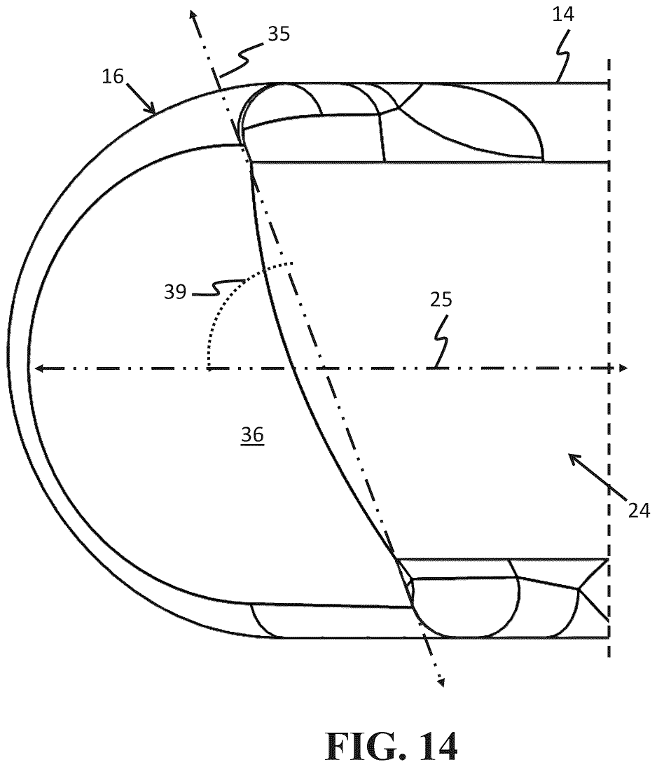

In some embodiments, and as shown with particular reference to FIG. 14, the contact surface plane 35 formed by the concave contact surface 36 of the foot 16 may form a contact surface angle 39 relative to the longitudinal axis 25 of the shaft 14. In some embodiments, the contact surface angle 39 is between about forty-five and about ninety degrees. In other embodiments, the contact surface angle 39 is between about sixty and about eighty degrees. In further embodiments, the contact surface angle 39 is about seventy degrees. As mentioned above with reference to FIG. 11, the rear surface plane 71 formed by the rear surface 70 of the handle 12 may define the rear surface angle 72 relative to the longitudinal axis 25 of the shaft 14. In some embodiments, the contact surface angle 39 and the rear surface angle 72 are equivalent. For example, in some embodiments, each of the rear surface angle 72 and the contact surface angle 39 may be approximately seventy degrees. In other words, the rear surface 70 may be parallel to the concave contact surface 36. The equivalence of the rear surface angle 72 and the contact surface angle 39 may aid a surgeon in ensuring concise control and proper alignment of the apparatus 100 during a procedure.

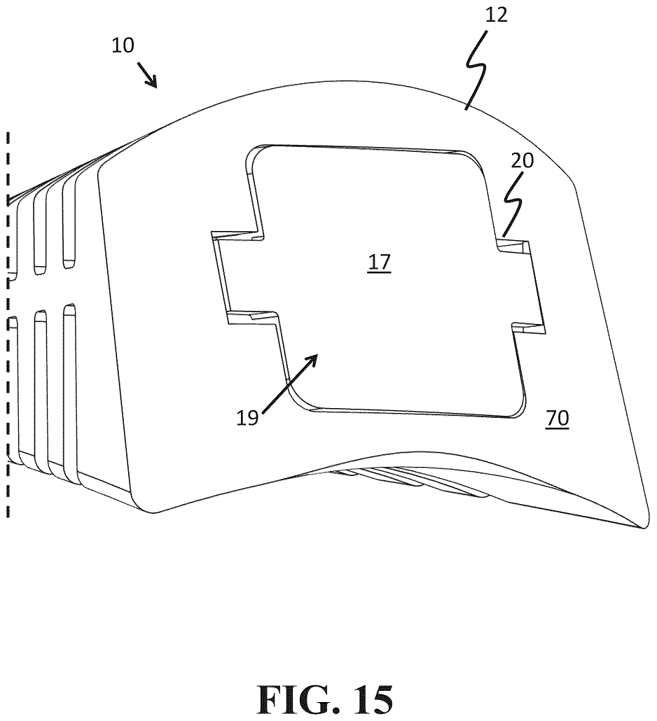

Referring to FIG. 15, a rear perspective view of the body 10 is shown, according to some embodiments of the present disclosure. As shown, the hub 19 may be received by the receiver 20 such that a rear hub surface 17 of the hub 19 is coplanar (or substantially coplanar) with the rear surface 70 of the handle 12. In some embodiments, the edges of the receiver 20 are contoured to match with corresponding edges of the hub 19, such that the hub 19 may only be inserted within the receiver 20 in a particular orientation.

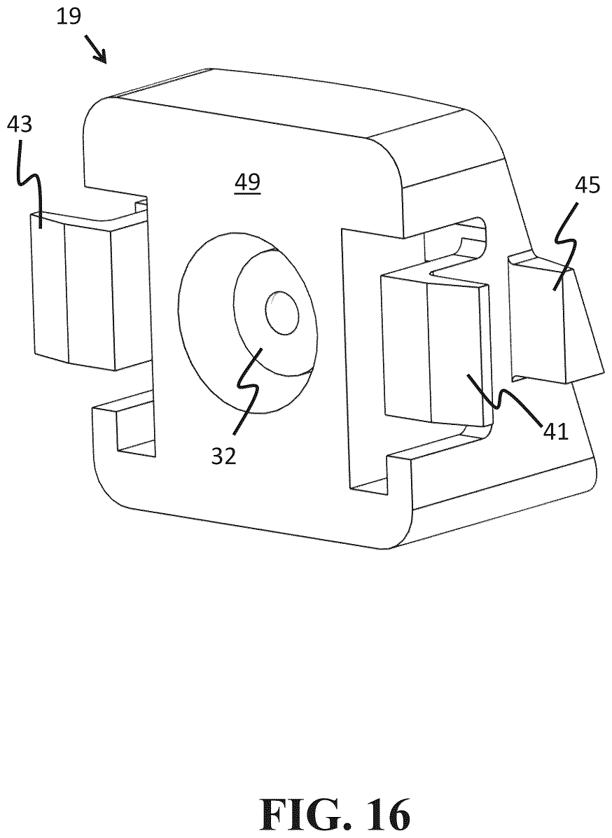

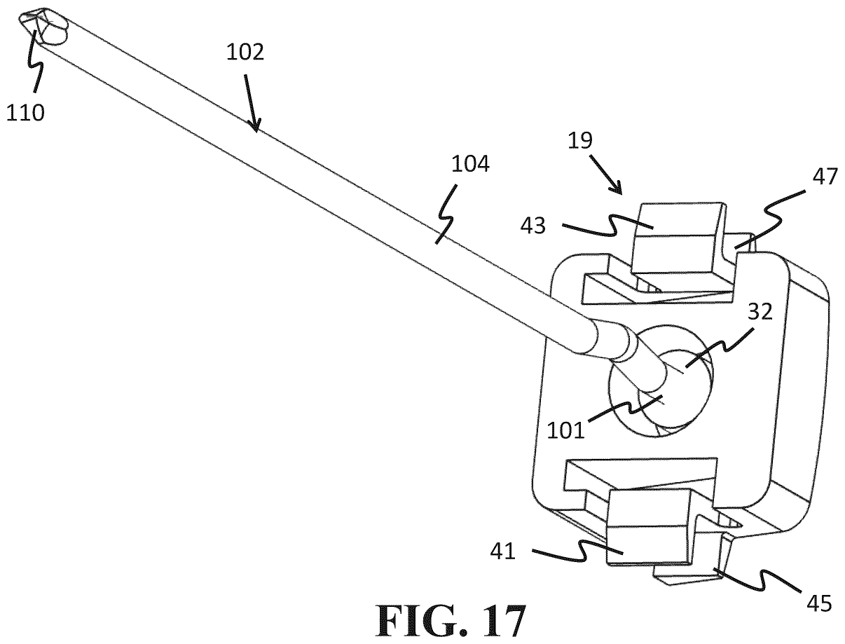

Referring to FIG. 16, a front perspective view of the hub 19 is shown, according to some embodiments of the present disclosure. In some embodiments, the hub 19 includes a front hub surface 49 opposite the rear hub surface 17 (shown with reference to FIG. 10). The hub 19 may include an instrument base 32, a first flange 41, a second flange 43 opposite the first flange 41, a first tab 45, and a second tab 47 (shown with reference to FIG. 17).

Referring to FIG. 17, a front perspective view of the cutting instrument 102 fixed to the hub 19 is shown, according to some embodiments of the present disclosure. The proximal end 101 of the cutting instrument 102 may be fixed to the instrument base 32 of the hub 19. Thus, the cutting instrument 102 may extend distally from the hub 19.

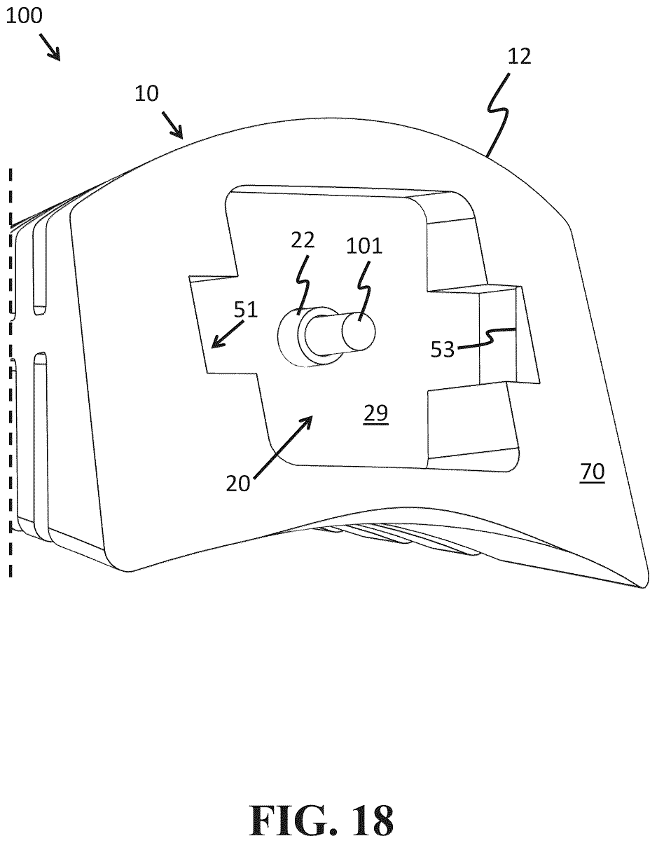

Referring to FIG. 18, a rear perspective view of the receiver 20 is shown, according to some embodiments of the present disclosure. As shown, the receiver 20 may include a port 22 through which the cutting instrument 102 may extend.

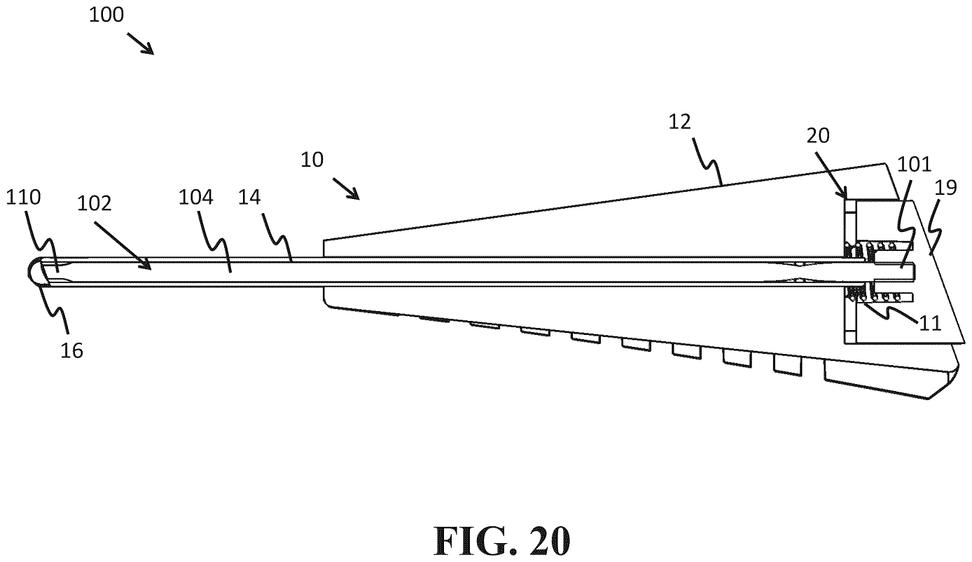

In some embodiments, the receiver 20 defines a first ledge 51 and a second ledge 53 opposite the first ledge 51. When the hub 19 is received within the receiver 20 (as shown with reference to FIG. 20), the first flange 43 may be ramped and flexible enough to bend and pass over the first ledge 51 and, similarly, the second flange 45 may be ramped and flexible enough to bend and pass over the second ledge 53. Of course, once the first and second flanges 43, 45 have passed over the first and second ledge 51, 53, the first and second flanges 43, 45 may prevent the hub 19 from being removed from the receiver 20. For instance, while the first and second flanges 43, 45 may be ramped on one side (e.g., the side facing the first and second ledges 51, 53 prior to insertion of the hub 19 within the receiver 20), the first and second flanges 43, 45 may not be ramped on the opposite side. In this sense, the first and second flanges 43, 45, in conjunction with the first and second ledges 51, 53, may allow the hub 19 to be inserted within the receiver 20, but prevent the hub 19 from being removed from the receiver 20 once inserted.

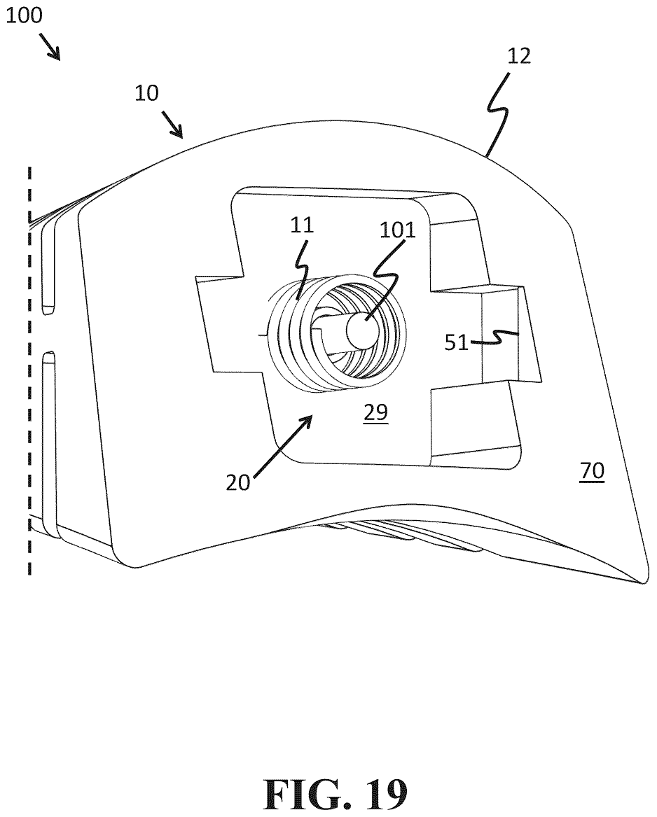

Referring now to FIG. 19, a biasing member 11 is shown, according to some embodiments of the present disclosure. The biasing member 11 is further depicted with reference to FIGS. 3, 4, and 11. The biasing member 11 may be disposed in the receiver 20. When the hub 19 is received by the receiver 20, the biasing member 11 may be disposed between the hub 19 and the handle 12 (as shown with reference to FIGS. 3, 4, and 11). The biasing member 11 may resist advancement of the hub 19 towards the handle 12 (or, more generally, the forward advancement of the cutting instrument 102).

In some embodiments, the biasing member 11 is a spring. In other embodiments, the biasing member 11 is some other member configured to resist the advancement of the hub 19 towards the handle 12. In some embodiments, such resistance is provided in the form of a resisted compression. In other embodiments, such resistance is provided in the form of a resisted expansion. In further embodiments, the apparatus 100 does not include the biasing member 11. For instance, advancement of the hub 19 towards the handle 12 may be controlled by a press-fit engagement between the receiver 20 and the hub 19 (e.g., such as angled corresponding surfaces on the inner surface of the receiver 20 and the outer surface of the hub 19), or some other suitable method of facilitating desirable resistance against advancement of the hub 19 towards the handle 12. In even further embodiments, no such resistance is directly provided, and the advancement of the hub 19 (or, the cutting instrument 102 in general) is controlled in some other suitable fashion.

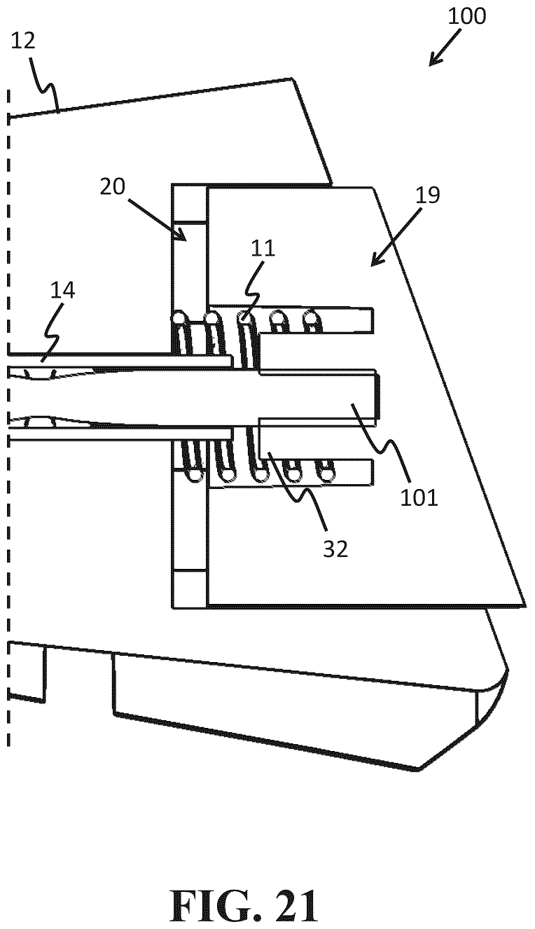

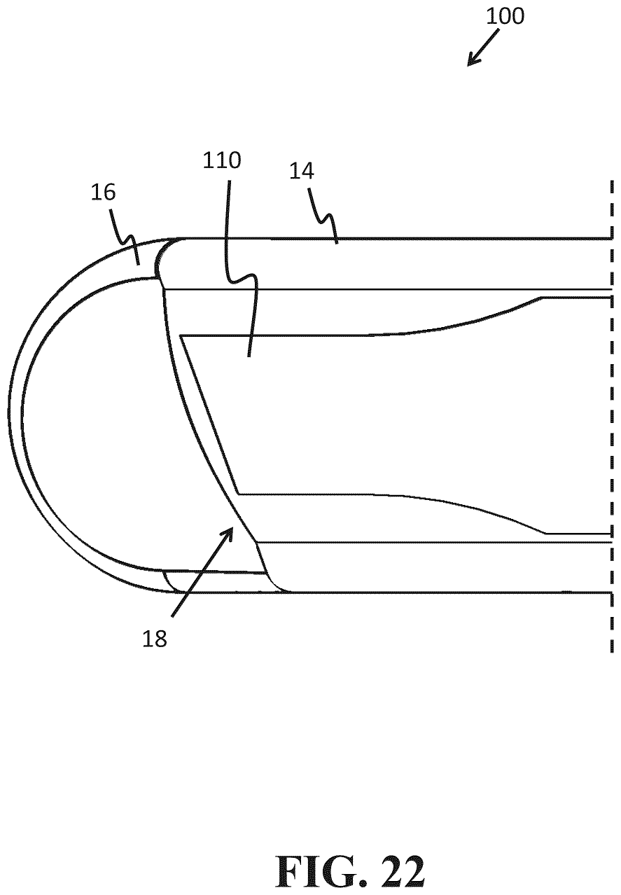

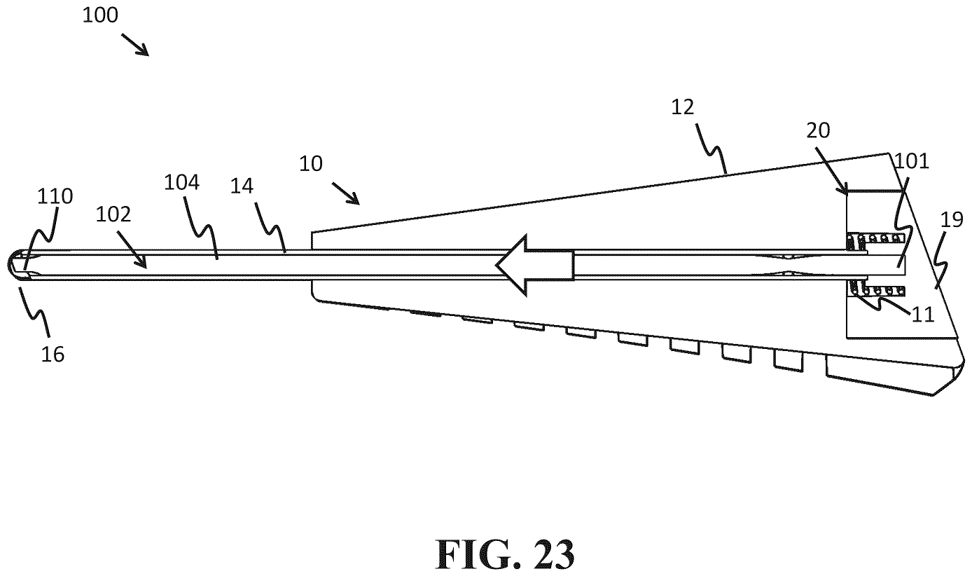

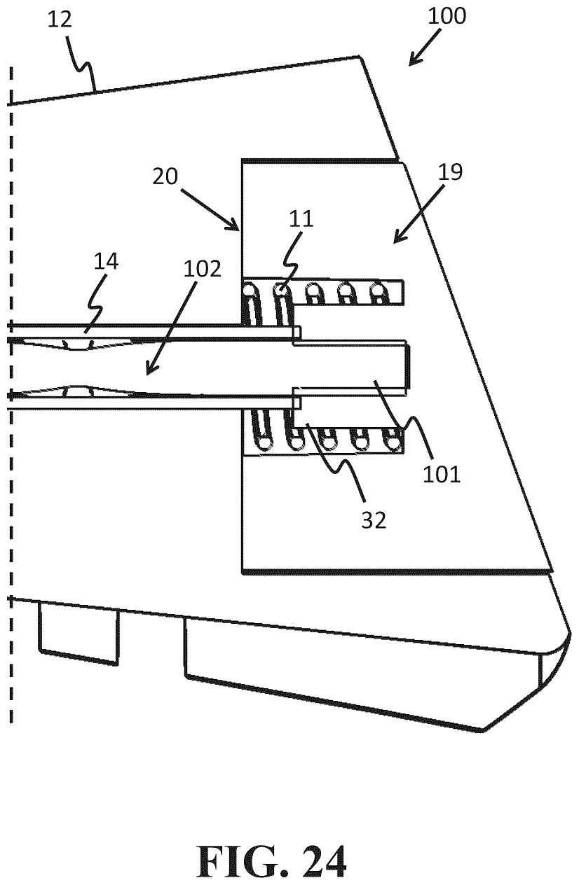

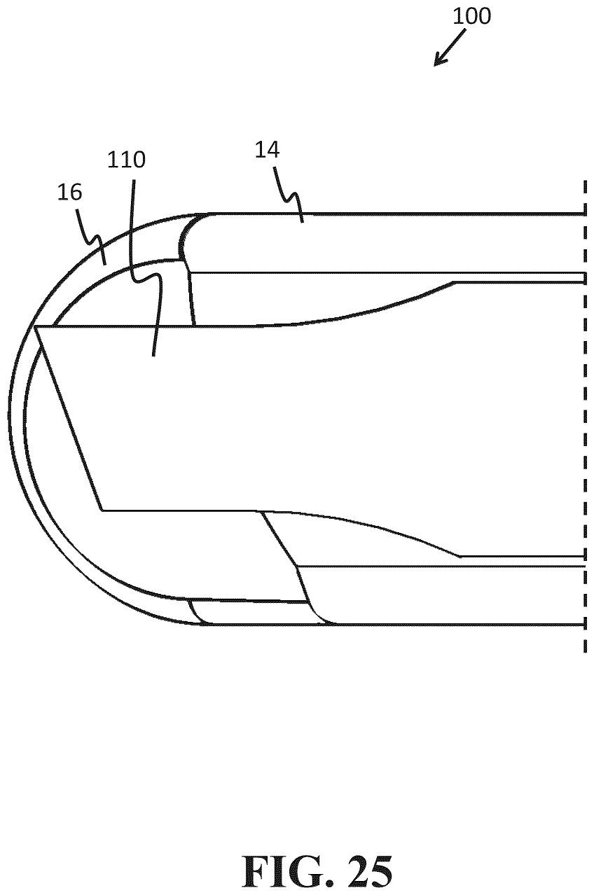

Referring to FIGS. 20-25, side cross-sectional views of the apparatus 100 are shown, according to some embodiments of the present disclosure. As shown, the hub 19 (with the cutting instrument 102 fixed thereto) may be inserted in the receiver 20, such that the cutting instrument 102 extends through the channel 24 and towards the foot 16. FIGS. 20-22 depict the apparatus 100 in a “resting” arrangement and FIGS. 23-25 depict the apparatus 100 in an “engaged” arrangement. In some embodiments, the biasing member 11 pushes the hub 19 away from the handle 12, until the first and second flanges 43, 45 of the hub 19 rest against inner surfaces of the first and second ledges 51, 53, which act to prevent the hub 19 from being pushed out of the receiver 20 entirely. The cutting instrument 102 may be dimensioned such that when the apparatus 100 is configured in the “resting” arrangement depicted with reference to FIGS. 20-22, the tip 110 of the cutting instrument 102 is positioned about the opening 18 of the foot 16, without projecting past the opening 18 of the foot 16. For example, the length of the cutting instrument 102 may be between about eight and nine centimeters. In further embodiments, the length of the cutting instrument is about 8.9 centimeters.

Referring particularly to FIGS. 23-25, the hub 19 may be pressed towards the handle 12 in order to transition the apparatus 100 from the “resting” arrangement to the “engaged” arrangement. Due to the resiliency of the biasing member 11, the cutting instrument 102 may exhibit a tendency in some applications to be biased axially away from foot 16 towards the handle 12. A user may overcome this bias by maintaining pressure against the hub 19 into the receiver 20 to keep the hub 19 fully seated in the receiver 20. For instance, a user may press the hub 19 (against the biasing force of the biasing member 11) until the front hub surface 49 of the hub 19 (shown with reference to FIG. 16) rests against an inner face 29 of the receiver 20 (shown with reference to FIG. 19). Pressing the hub 19 towards the handle 12, in turn, may distally advance the cutting instrument fixed thereto. In this sense, the length of the cutting instrument 102 may be dimensioned such that when the hub 19 bottoms out in the receiver 20, the tip 110 protrudes distally from the foot 16.

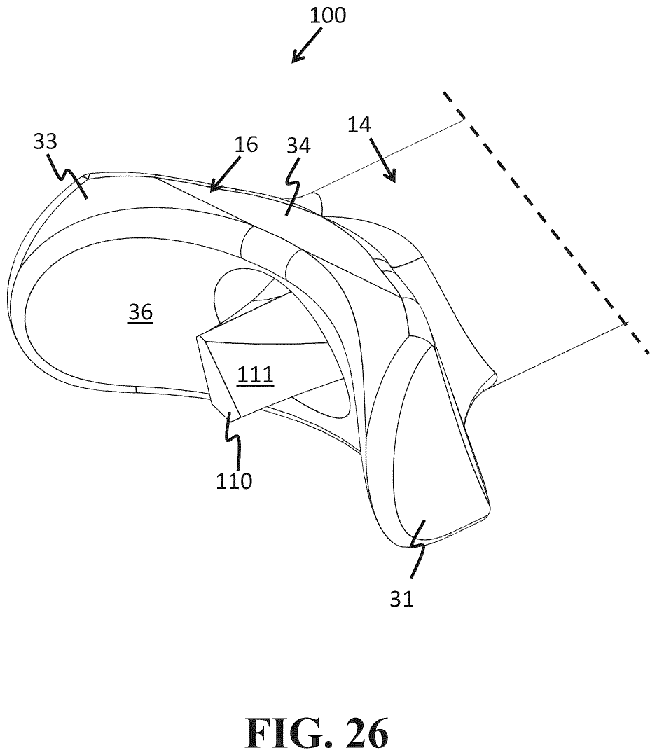

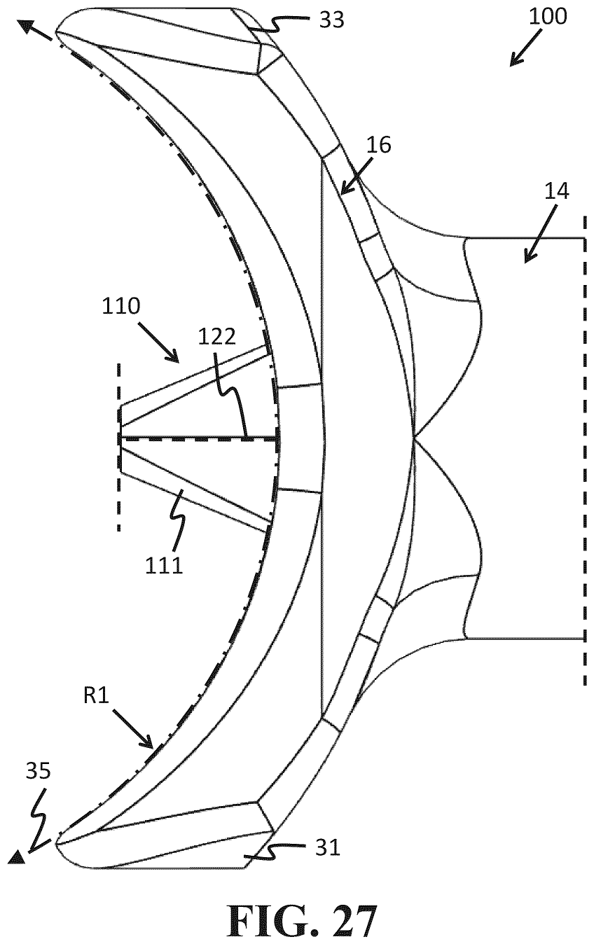

When the hub 19 has been pressed towards the handle 12 such that the apparatus 100 is configured in the “engaged” arrangement, the tip 110 may project distally from the opening 18 of the foot 16, as discussed in greater detail below with reference to FIGS. 26-27. The apparatus 100 may be used to cut tissue when configured in the “engaged” arrangement. During a procedure, a user may release the pressure from the hub 19 to automatically withdraw the tip 110 back into the opening 18. In this sense, the biasing force of the biasing member 11 may require that the operator maintain a constant pressure against the hub 19, and whenever such pressure is released, the apparatus 100 may then return to the “resting” arrangement. As such, the biasing fitment between the hub 19 and the receiver 20 may provide a safety mechanism allowing a surgeon to quickly and easily retract the tip 110 by releasing the pressure against hub 19. This, in turn, may reduce the likelihood of inadvertent cuts made by the tip 110 of the cutting instrument 102. Furthermore, as mentioned above, the apparatus 100 as disclosed herein may be disposable. Advantageously, the automatic return of the apparatus 100 to the “resting” arrangement mentioned above makes the apparatus 100 more suitable for disposal (e.g., by returning the tip 110 into the channel 24 and avoiding a sharp edge on the apparatus 100 following use that may otherwise inhibit safe disposal).

Referring to FIGS. 26 and 27, the tip 110 is shown projecting from the opening 18 of the foot 16, according to some embodiments of the present disclosure. For instance, the tip 110 may project from the opening 18 when the apparatus 100 has been configured in the “engaged” arrangement as discussed above. As shown with particular reference to FIG. 27, in such arrangements, the tip 110 may achieve a cutting clearance 122 relative to the foot 16 (or, more particularly, the concave contact surface 36 about the longitudinal axis 25). For example, the cutting clearance 122 may be defined as the maximum distance (measured along the longitudinal axis 25) that the tip 110 clears the concave contact surface 36 of the foot 16 (or the contact surface plane 35 formed thereby).

In some embodiments, lateral ends of the foot 16 may extend relative to the opening 18 of the foot 16. For example, the concave contact surface 36 may form first and second lips 31, 33 on opposing lateral ends of the foot 16 that extend distally relative to the opening 18. The cutting clearance 122 may be configured such that when the apparatus 100 is configured in the “engaged” arrangement, the tip 110 of the cutting instrument 102 projects distally out of the foot 16 and is prevented from extending further from the opening 18 than the first and second lips 31, 33. In this sense, when the hub 19 is advanced toward the foot 16 in order to configure the apparatus 100 in the “engaged” arrangement, the tip 110 of the cutting instrument 102 may project distally out of the opening 18 of the foot 16 but be prevented from extending further from the opening 16 than the opposing lateral ends of the foot 16 (e.g., the first and second lips 31, 33). Accordingly, the foot 16 may serve as a soft tissue dissector tool, retractor tool, and a safety mechanism by limiting the depth of the deployed cutting instrument and thus preventing damage to a flexor tendon.

In some embodiments, the cutting clearance 122 may be between about one and about two millimeters. In some embodiments, the cutting clearance 122 is between about 1.4 and about 1.6 millimeters. In further embodiments, the cutting clearance 122 is about 1.5 millimeters. Depending on the implementation, the cutting clearance 122 may be adjusted (whether by providing a longer tube 104, a shorter shaft 14, or some other dimensional variation) to achieve optimal performance of the apparatus 100. Advantageously, the cutting clearance 122 may be configured such that the tip 110 is only allowed to extend so far as to sever the A1 pulley without damaging the underlying tendon, as discussed herein, without causing inadvertent damage to the hand.

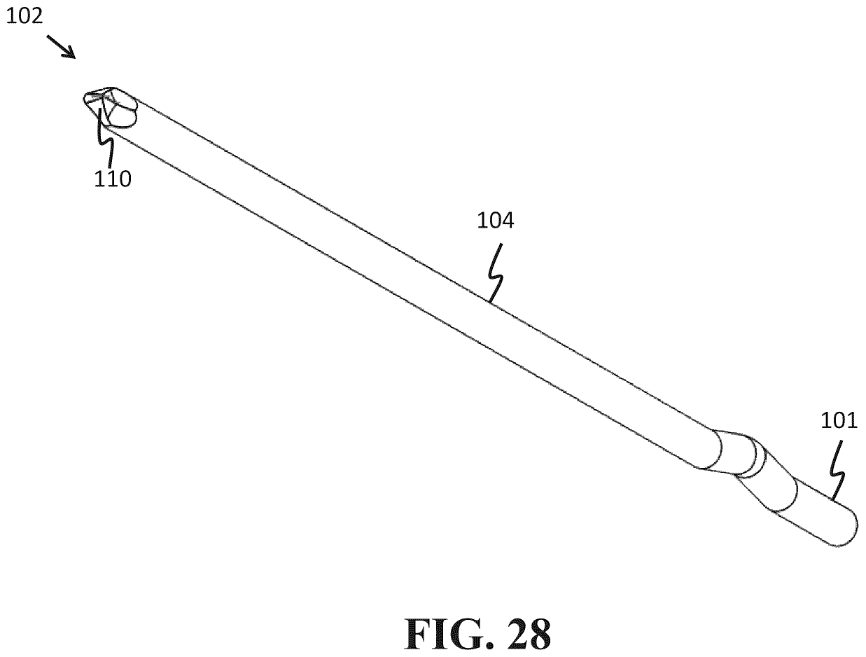

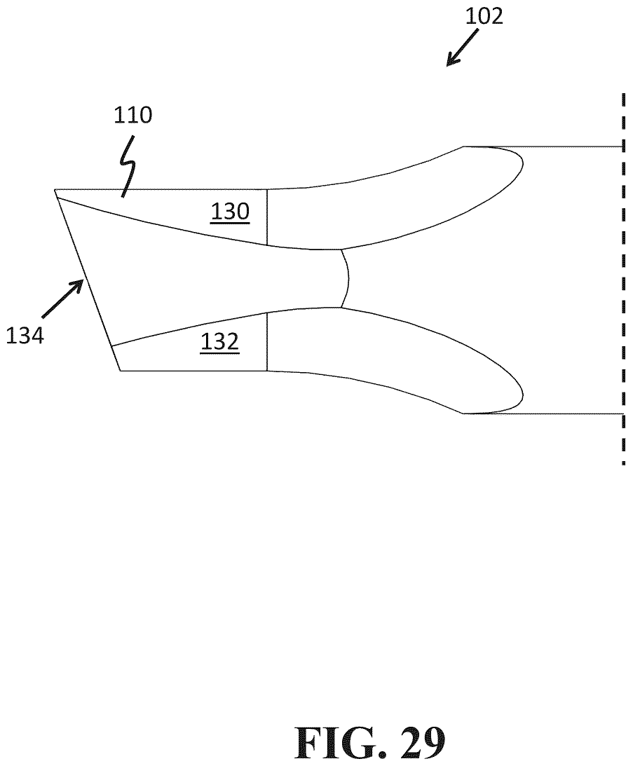

Referring to FIGS. 28-30, the cutting instrument 102 is shown in greater detail, according to some embodiments of the present disclosure. In some embodiments, the tip 110 includes a sharp point that may be used to cut tissue by moving the needle tip forward and backwards or from side to side to dissect tissue. In other words, the tip 110 may be configured to dissect tissue during a surgical procedure.

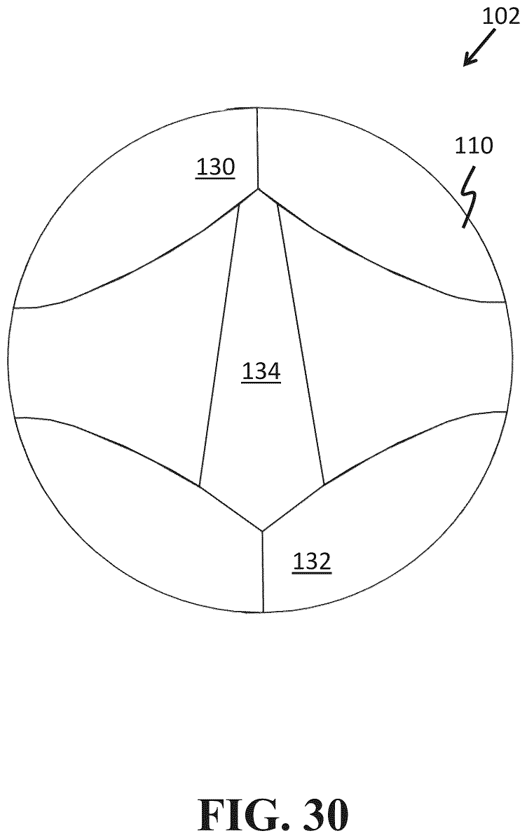

In some embodiments, and as shown with particular reference to FIG. 30, the tip 110 includes an anterior cutting surface 130, a posterior cutting surface 132, and a blunt end 134. For instance, the blunt end 134 may advantageously prevent rest against a tendon during a procedure and avoid cutting the tendon, thereby avoiding inadvertent damages to tissue during the procedure. In other embodiments, the tip 110 includes a beveled surface.

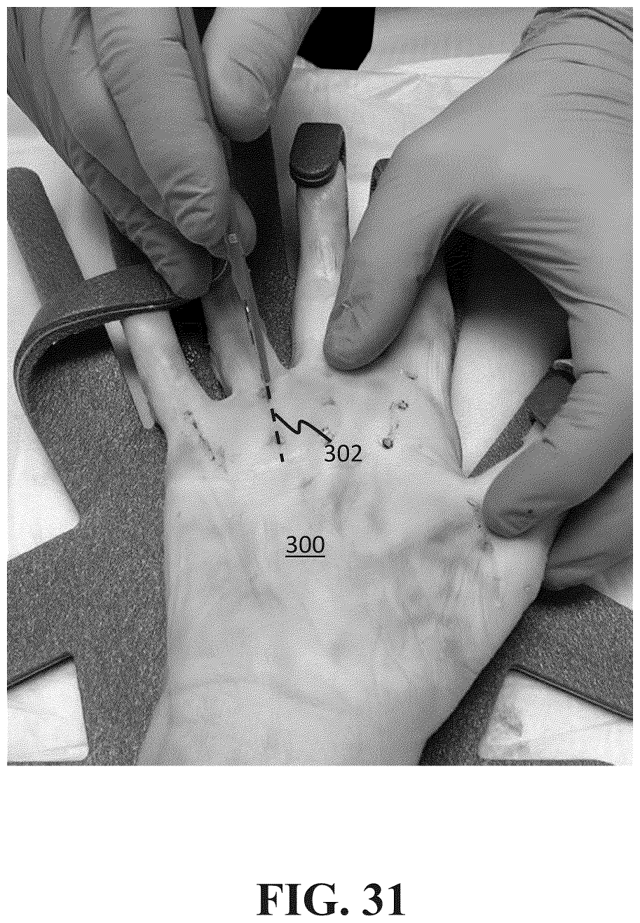

Referring now to FIGS. 31-35, an exemplary method of treatment of a trigger digit using the apparatus 100 is shown, according to some embodiments of the present disclosure. As shown with reference to FIG. 31, a small incision near a tendon sheath 302 may be made in a palm of a hand 300.

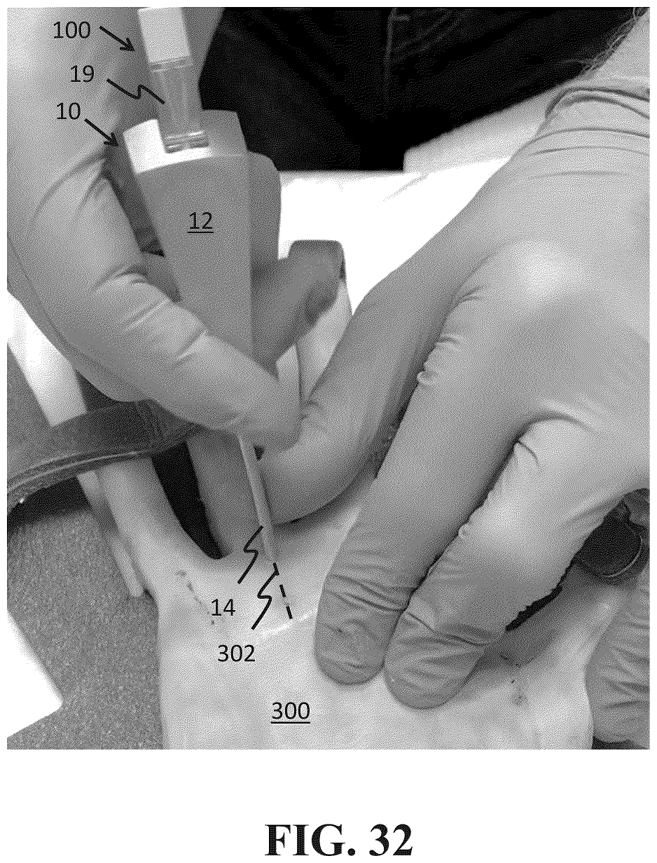

As shown with reference to FIG. 32, the foot 16 of the apparatus 100 may be inserted within the incision site, such that the foot 16 is seated over the tendon sheath 302. As shown, once the foot 16 has been inserted within the incision site, the foot 16 may not be entirely visible to the surgeon. However, as discussed above, the shapes and contours of the apparatus 100 may aid the surgeon in centering the foot 16 on the tendon sheath 302. As examples, and as further discussed above, the equivalent arcs formed by the concave contact surface plane 35 and the rear surface plane 71, as well as the equivalence of the rear surface angle 72 and the contact surface angle 39, may aid the surgeon in centering the foot 16 on the tendon sheath 302. Once the foot 16 has been centered on the tendon sheath 302, the foot 16 may be maneuvered about the tendon sheath 302. For example, and as discussed above, each of the first and second lips 31, 33 of the foot 16 may sweep soft tissue from opposing sides from the surface of the tendon sheath.

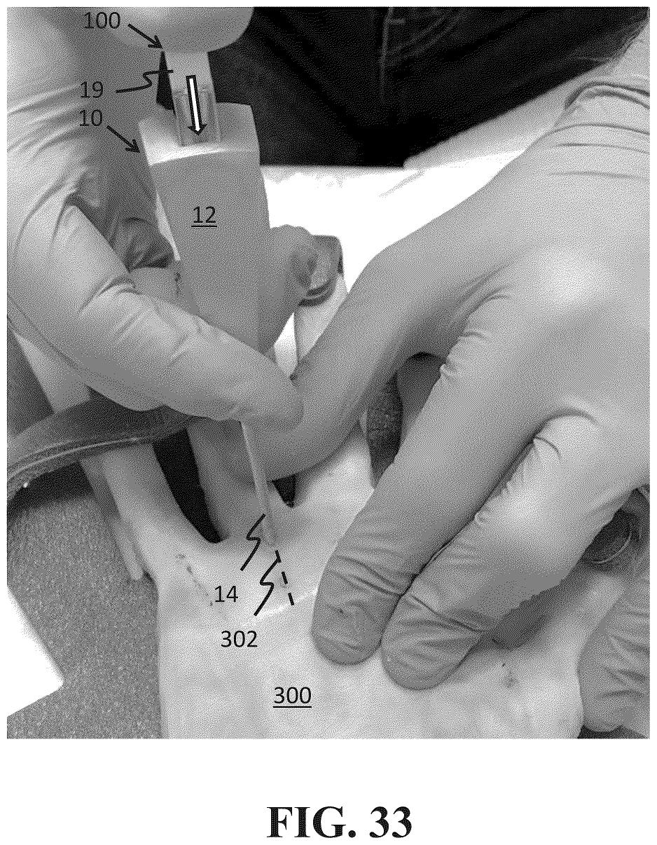

When the foot 16 of the apparatus 100 is inserted within the incision site as shown with reference to FIG. 32, the apparatus 100 may be configured in the “resting” arrangement as discussed above. As shown with reference to FIG. 33, the surgeon may then press the hub 19 into the receiver 20 until the hub 19 is seated within the receiver 20 as discussed herein (configuring the apparatus 100 into the “engaged” arrangement as discussed above), such that the cutting instrument 102 is further advanced through the channel 24 and the tip 110 projects from the foot 16 to make an incision in the A1 pulley.

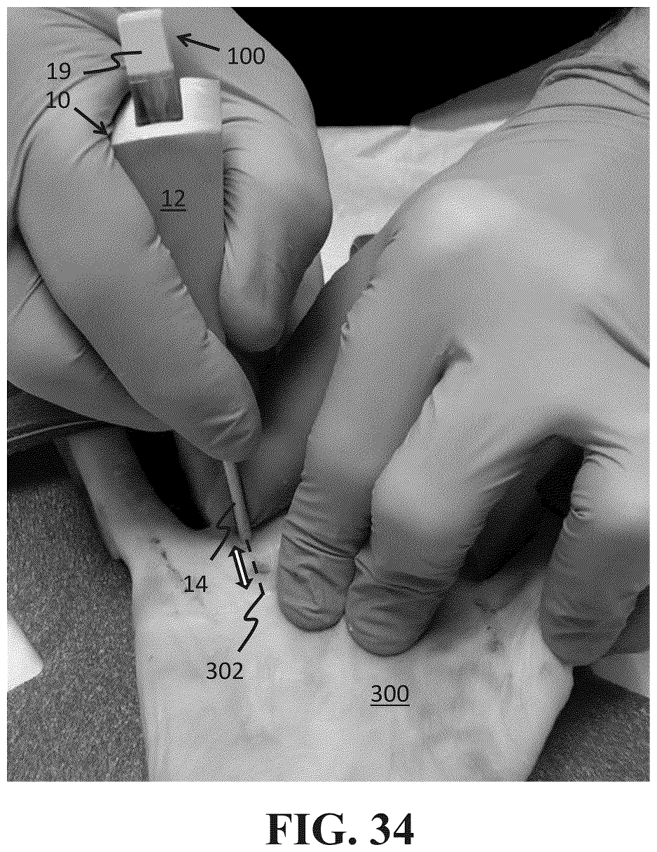

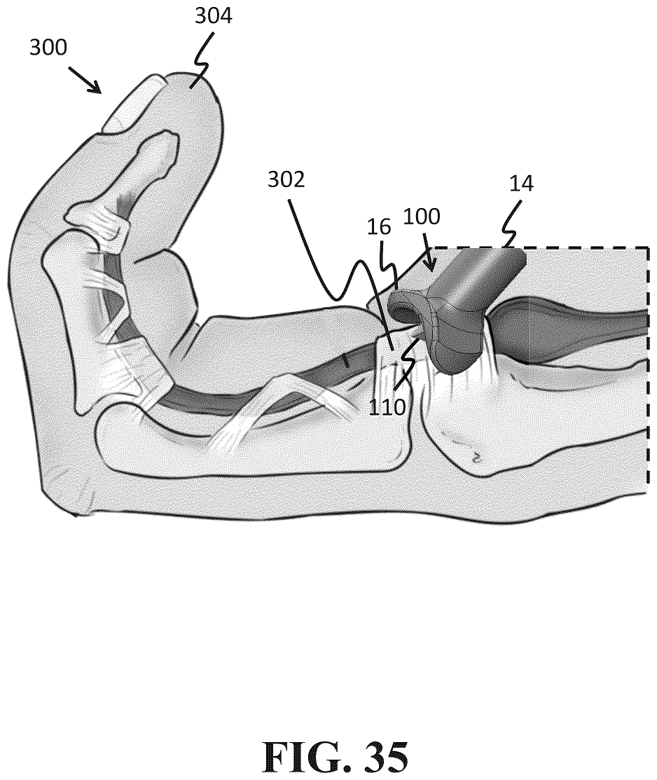

As shown with reference to FIGS. 34 and 35, the tip 110 of the cutting instrument 102 may then be advanced to dissect tissue (e.g., make appropriate cuts on the A1 pulley) by manually controlling the handle in a back and forth or side to side motion. For example, and as shown with particular reference to FIG. 35, the tip 110 of the cutting instrument 102 may be advanced to make appropriate cuts on an A1 pulley 302 of a finger 304 on the hand 300.

Accordingly, the present disclosure further provides for a method. The method may include a first step of providing the handle 12, the shaft 14 extending distally from the handle 12, and the foot 16 disposed on a distal end of the shaft 14 opposite the handle 12. The method may further include a second step of positioning the foot 16 on the tendon sheath 302. As discussed above, the foot 16 may include the concave contact surface 36 and the opening 18 disposed on the concave contact surface 36, where the concave contact surface 36 is shaped to accommodate the tendon sheath 302. The method may further include a third step of advancing the cutting instrument 100 through the channel 24 defined between the port 22 and the opening 18, such that the tip 110 of the cutting instrument 100 projects distally out of the opening 18 and is operable to dissect tissue during a surgical procedure.

Accordingly, further embodiments of the present disclosure include a method of performing surgery. The method may include providing a surgical tool including the aforementioned handle 12, shaft 14, foot 16, cutting instrument 102, and hub 19. The method may further include positioning the foot 16 on the tendon sheath and advancing the hub 19, such that the tip 110 of the cutting instrument 102 projects distally out of the opening 18 of the foot 16 and is operable to dissect tissue during the surgical procedure. Thus, once aligned with an A1 pulley in a patient's hand, surgical tool may be manipulated such that the tip 110 of the cutting instrument 102 is used to cut the tissue at a desired location. The entire procedure may be performed with or without ultrasound imaging in a doctor's office.

Thus, although there have been described particular embodiments of the present invention of a new and useful APPARATUS AND METHODS FOR TRIGGER DIGIT RELEASE, it is not intended that such references be construed as limitations upon the scope of this invention.

What is claimed is:

1. An apparatus, comprising:
a handle;
a shaft extending distally from the handle;
a receiver defined in a proximal end of the handle opposite the shaft;
a hub disposed in the receiver;
a foot disposed on a distal end of the shaft opposite the handle and including an opening;
a channel defined from the receiver to the opening; and
a cutting instrument disposed in the channel, fixed to the hub, and including a tip that is configured to dissect tissue during a surgical procedure.

2. The apparatus of claim 1, wherein the handle includes a series of ribs spaced longitudinally along the length of the handle.

3. The apparatus of claim 1, wherein the handle is made of either plastic or a polymer material.

4. The apparatus of claim 1, wherein each of the handle, shaft, hub, and foot are made of either plastic or a polymer material.

5. The apparatus of claim 1, wherein the foot includes a concave contact surface shaped to accommodate a tendon sheath, and
wherein the opening is disposed on the concave contact surface.

6. The apparatus of claim 1, further comprising a biasing member disposed in the receiver between the hub and the handle.

7. The apparatus of claim 1, wherein opposing lateral ends of the foot extend distally relative to the opening, and
wherein when the hub is advanced toward the foot, the tip of the cutting instrument projects distally out of the opening of the foot and is prevented from extending further from the opening than the opposing lateral ends of the foot.

8. A method, comprising:
providing a handle including a receiver defined in a proximal end of the handle, a shaft extending distally from the handle opposite the receiver, a foot disposed on a distal end of the shaft opposite the handle and including an opening, a hub disposed in the receiver, and a cutting instrument fixed to the hub and disposed in a channel defined from the receiver to the opening;
positioning the foot on a tendon sheath; and
advancing the hub, such that a tip of the cutting instrument projects distally out of the opening and is operable to dissect tissue during a surgical procedure.

9. The method of claim 8, wherein the handle includes a series of ribs spaced longitudinally along the length of the handle.

10. The method of claim 8, wherein the handle is made of either plastic or a polymer material.

11. The method of claim 8, wherein each of the handle, shaft, hub, and foot are made of either plastic or a polymer material.

12. The method of claim 8, wherein the foot includes a concave contact surface shaped to accommodate the tendon sheath, and
wherein the opening is disposed on the concave contact surface.

13. The method of claim 8, wherein a biasing member disposed in the receiver between the hub and the handle resists the advancement of the hub.

14. The method of claim 8, wherein opposing lateral ends of the foot extend distally relative to the opening, such that when the hub is advanced, the tip of the cutting instrument is prevented from extending further from the opening than the opposing lateral ends of the foot.

15. An apparatus, comprising:
a handle including a series of ribs spaced longitudinally along the length of the handle;
a shaft extending distally from the handle;
a receiver defined in a proximal end of the handle opposite the shaft;
a foot disposed on a distal end of the shaft opposite the handle and including an opening;
a channel defined from the receiver to the opening; and
a cutting instrument disposed in the channel and including a tip that is configured to dissect tissue during a surgical procedure.

16. The apparatus of claim 15, wherein the handle is made of plastic.

17. The apparatus of claim 16, wherein each of the shaft and foot are made of plastic.

18. The apparatus of claim 15, wherein the handle is made of a polymer material.

17. The apparatus of claim 18, wherein each of the shaft and foot are made of the polymer material.

19. The apparatus of claim 15, wherein the foot includes a concave contact surface shaped to accommodate a tendon sheath, and
wherein the opening is disposed on the concave contact surface.

20. The apparatus of claim 15, wherein opposing lateral ends of the foot extend distally relative to the opening, and
wherein when the hub is advanced toward the foot, the tip of the cutting instrument projects distally out of the opening of the foot and is prevented from extending further from the opening than the opposing lateral ends of the foot.
